(19) United States
(12) Patent Application Publication
(10) Pub. No.: US 2025/0146730 A1
(43) Pub. Date: May 8, 2025

(54) SINGLE USE HEAT EXCHANGER AND DEVICES INCORPORATING THE SAME

(71) Applicant: AQUASYN, LLC, Carson City, NV (US)

(72) Inventors: Michael C. Gagne, Carson City, NV (US); Luke Davis, Carson City, NV (US); Loren B. Montague, Carson City, NV (US)

(73) Assignee: AQUASYN, LLC, Carson City, NV (US)

(21) Appl. No.: 18/838,058

(22) PCT Filed: Feb. 6, 2023
(86) PCT No.: PCT/US2023/062080

§ 371 Date: Aug. 13, 2024

Related U.S. Application Data
(60) Provisional application No. 63/309,974, filed on Feb. 14, 2022.

(51) Int. Cl. F25D 5/00

(52) U.S. Cl. CPC F25D 5/00

(57) ABSTRACT

A single use heat exchanger device includes an outer bag formed from a flexible polymer film and defining an interior compartment, the outer bag having a first port and a second port disposed therein to provide fluid access to and from the interior compartment. An inner bag is formed from a flexible polymer film and defines an interior flow path between a third port and a fourth port, the third port and the fourth port extending through the outer bag and provide fluid access to and from the interior flow path. In other embodiments, a flexible bag or container incorporates heat exchanger flow path(s) into one or more surfaces. In another embodiment, a container or vessel incorporates heat exchange flow path around a cylindrical surface formed by an inner jacket and outer jacket.

### RELATED APPLICATION

This Application claims priority to U.S. Provisional Patent Application No. 63/309,974 filed on Feb. 14, 2022, which is hereby incorporated by reference in its entirety. Priority is claimed pursuant to 35 U.S.C. § 119 and any other applicable statute.

### TECHNICAL FIELD

The technical field generally relates to heat exchangers used in connection with biopharmaceutical, pharmaceutical, and biotechnology applications. More specifically, the technical field relates to single use heat exchangers.

### BACKGROUND

Biopharmaceutical, pharmaceutical, and biotechnology applications have historically used reusable devices in the research and manufacturing processes associated with these industries. These reusable devices, however, must be cleaned and sterilized between uses. This was typically accomplished using a sterilization agent (e.g., steam) that was introduced into the reusable devices. The sterilization process, however, is time-consuming and introduces considerable cost into the process. Moreover, there is the possibility that sterilization was not effective, leading to contamination issues. More recently, these industries are moving toward using single use components instead of reusable devices for parts and components that come into contact with reagents and products. An advantage of single use or disposable products is that it eliminates the need for cleaning and sterilizing devices in between uses.

Heat exchangers are one such device that are commonly used in biopharmaceutical, pharmaceutical, and biotechnology applications. Heat exchangers are used to heat or cool (or maintain temperatures) fluids that are used in the manufacturing process. For example, cells used in biotechnology applications may need to be grown or maintained in a relatively narrow range of temperatures and fluids that interact with the cells may need to be cooled or heated as appropriate. Currently, there exists single use heat exchangers that are used in connection with a larger plate-based system to heat or cool fluids. For example, the DHX™ heat exchanger made by ThermoFisher Scientific is a modular heat exchanger system that uses four single-use bioprocess fluid containers as the sterile fluid path. The bioprocess fluid containers are fit tightly between several stainless-steel plates that are used to transfer heat to and from the bioprocess fluid containers. This heat exchanger, while using some single-use components, requires a large and bulky system of plates that are used to sandwich the bioprocess fluid containers. This makes the entire system bulky and expensive. Alternative single use heat exchangers and devices incorporating such heat exchangers are needed.

### SUMMARY

In one embodiment, a single use heat exchanger device includes an outer bag and an inner bag. The outer bag is formed from a flexible polymer film and defines an interior compartment, the outer bag having a first port and a second port disposed therein to provide fluid access to and from the interior compartment. The inner bag is formed from a flexible polymer film and defines an interior flow path that extends between a third port and a fourth port that extend through the outer bag and provide fluid access to and from the interior flow path of the inner bag. Fluid contained in the interior compartment of the outer bag surrounds the inner bag and is able to exchange heat to or from the fluid contained in the interior flow path of the inner bag. One of the first or second ports is used to flow a heat exchange fluid into the interior compartment of the outer bag while the other port is used to remove the heat exchange fluid. Fluid containing product or reagents is flowed into one of the third or fourth ports of the inner bag and is removed from the inner bag via the other port. The heat exchange fluid surrounds the interior flow path (or multiple such paths) to exchange heat between respective fluids carried by the inner and outer bags.

In another embodiment, the single use heat exchanger device includes an outer bag formed from a flexible polymer film and defining an interior compartment, the outer bag having a first port and a second port disposed therein to provide fluid access to and from the interior compartment. The heat exchanger includes an inner bag formed from a flexible polymer film and defining an interior flow path that extends between a third port and a fourth port that provide fluid access to and from the interior flow path of the inner bag, wherein the third port and the fourth port are suspended by a standoff from a separate fluid passageway located in the first port and second port. The standoff may be a tube that connects the inner bag port to the outer bag port. For example, barbed nipples on each port may be connected using a segment of tubing used as the standoff.

In another embodiment, a single use heat exchanger device includes a flexible bag or container having one or more surfaces and defining an interior compartment, the flexible bag or container having one or more inlet ports and one or more outlet ports disposed therein to provide fluid access to and from the interior compartment wherein one or more of the surfaces of the flexible bag or container comprises a heat exchanger flow path contained thereon, wherein each heat exchanger flow path comprises a heat exchange fluid inlet port and outlet port. The heat exchanger flow path(s) may take a number of configurations, shapes, and sizes. Examples include s-coil shapes and dimpled surfaces. Products or reagents are flowed in or contained in the interior compartment of the flexible bag or container while heat exchange fluid is flowed into the heat exchange fluid inlet port and runs through the heat exchanger flow path(s) and exits the heat exchange fluid outlet port.

In another embodiment, a container or vessel having an integrated heat exchanger includes an inner jacket comprising one or more polymer films or layers and an outer jacket comprising one or more polymer films or layers. A first fitting is located at one end of the inner jacket and the outer jacket and includes an inlet, the first fitting further having a first radial surface secured to the inner jacket and a second radial annular surface secured to the outer jacket. A second fitting is located at an opposing end of the inner jacket and the outer jacket and includes an outlet, the second fitting further having a first radial surface secured to the inner jacket and a second radial surface secured to the outer jacket. At least one heat exchange fluid inlet port is disposed in the first fitting and communicates with a fluid passageway contained in the first fitting that fluidically communicates with a space between the inner jacket and the outer jacket. At least one heat exchange fluid outlet port is disposed in the second fitting and communicates with a fluid passageway contained in the second fitting that fluidically communicates with the space between the inner jacket and the outer jacket.

In another embodiment, a method of manufacturing a single use heat exchanger includes providing a first flexible polymer film layer having apertures formed therein for receiving a first port, a second port, a third port, and a fourth port. A second flexible polymer film layer is placed on top of the first flexible polymer layer, the second flexible polymer film layer having apertures formed therein for the third port and fourth port. Next, a third flexible polymer film layer is placed on top of the second flexible polymer layer followed by placing a fourth flexible polymer film layer on top of the third flexible polymer layer. The first flexible polymer layer is welded to the fourth flexible polymer layer to form an outer bag. The second flexible polymer layer is welded to the third flexible polymer layer to form an inner bag that has the separate interior flow paths defined by welds. The first port and the second port are secured into respective apertures in the outer bag. The third port and fourth port are secured into respective apertures in the inner bag and outer bag. The first, second, third, and fourth ports may be secured using welds, adhesive, or a mechanical connection.

The laser welding may be automatically performed using, for example, a robotically controlled laser head. The various flexible polymer layers may be placed on a table or surface on which the laser welding takes place (e.g., welding table). The table or surface may contain apertures to receive the ports that are loaded into the inner and/or outer bags during the manufacturing process. In one embodiment, the third port and the fourth port each comprise separate welds to the first flexible polymer layer and the second flexible polymer layer. For example, the weld to the first flexible polymer layer may be disposed on an inner flanged surface of the third port and the fourth port and the weld to the second flexible polymer layer may be disposed on an outer flanged surface of the third port and the fourth port.

### BRIEF DESCRIPTION OF THE DRAWINGS

FIG. 1 illustrates a single use heat exchanger that includes an inner bag and an outer bag. The textured region of the inner bag represents a fluid contained therein.

FIG. 2A illustrates a perspective view of a single use heat exchanger device includes an outer bag and an inner bag.

FIG. 2B illustrate another perspective of a single use heat exchanger device includes an outer bag and an inner bag.

FIG. 2C illustrates a side view of a single use heat exchanger device includes an outer bag and an inner bag.

FIG. 3 illustrates an exploded view of the four flexible polymer layers that are used to form the single use heat exchanger device.

FIG. 4 illustrates a first flexible polymer layer that is placed on a welding table. Welding solution is applied around the periphery and around the apertures defined for the first port, second port, third port, and fourth port.

FIG. 5 illustrates a second flexible polymer layer that is placed in the first flexible polymer layer.

FIG. 6 illustrates welding solution that is applied to the exposed face of the second flexible polymer layer around the periphery and along segments that will form the separate interior flow paths.

FIG. 7 illustrates the first layer and the second layer with a first port, second port, third port, and fourth port inserted into the apertures for the first port, second port, third port, and fourth port.

FIG. 8 illustrates a cross-sectional view of the welding table used for laser welding with a fixture shown secured to the underside of the table to receive a port (the third port is illustrated but other ports have similar fixtures).

FIG. 9 illustrates a cross-sectional view of the third port that is inserted in the apertures of the first flexible polymer layer and the second flexible polymer layer. The third port includes a flanged end. An inner weld secures the first flexible layer to the third port and a separate outer weld secures the second flexible layer to the third port. The fourth port is secured in a similar manner.

FIG. 10 illustrates another embodiment of a single use heat exchanger that uses a standoff that suspends the inner bag from the outer bag via ports located in the outer bag.

FIGS. 11A-11C illustrate an alternative embodiment of a flexible bag or container that incorporates a heat exchange flow path into one or more sides or surfaces of a flexible bag or container. The heat exchange flow path has an s-coil shape that traverses the sides of the flexible bag or container. In this embodiment, two such sides or surfaces of a flexible bag or container have separate heat exchange flow paths integrated therein. FIG. 11A shows a side view of the flexible bag or container. FIG. 11B shows an end view of the flexible bag or container. FIG. 11C shows a perspective view of the flexible bag or container.

FIGS. 12A-12C illustrate an alternative embodiment of a flexible bag or container that incorporates a heat exchange flow path into one or more sides or surfaces of a flexible bag or container. The heat exchange flow path has an s-coil shape that traverses the sides of the flexible bag. In this embodiment, two opposing sides or surfaces of a flexible bag have separate heat exchange flow paths integrated therein. FIG. 12A shows a side view of the flexible bag. FIG. 12B shows an end view of the flexible bag. FIG. 12C shows a perspective view of the flexible bag. The flexible bag in this embodiment is a tapered flexible bag that has a common edge with surfaces extending therefrom that flare outward to define the interior volume of the bag.

FIGS. 13A-13C illustrate an alternative embodiment of a flexible bag or container that incorporates a heat exchange flow path into one or more sides or surfaces of a flexible bag or container. The heat exchange flow path includes a dimpled surface that extend across the sides of the flexible bag or container. In this embodiment, two opposing sides or surfaces of a flexible bag or container have separate heat exchange flow paths integrated therein. FIG. 13A shows a side view of the flexible bag or container. FIG. 13B shows an end view of the flexible bag or container. FIG. 13C shows a perspective view of the flexible bag or container.

FIGS. 14A-14C illustrate an alternative embodiment of a flexible bag or container that incorporates a heat exchange flow path into one or more sides or surfaces of a flexible bag or container. The heat exchange flow path includes a dimpled surface that extend across the sides of the flexible bag. In this embodiment, two opposing sides or surfaces of a flexible bag have separate heat exchange flow paths integrated therein. FIG. 14A shows a side view of the flexible bag. FIG. 14B shows an end view of the flexible bag. FIG. 14C shows a perspective view of the flexible bag. The flexible bag in this embodiment is a tapered flexible bag that has a common edge with surfaces extending therefrom that flare outward to define the interior volume of the bag.

FIG. 15A illustrates a perspective view of one embodiment of a container or vessel that incorporates a heat exchange flow path along a peripheral surface. The container or vessel is cylindrically shaped and has a side wall that is formed by a first layer or film. A second layer or film that is disposed radially outward of the first layer or film defines the heat exchange flow path. The container or vessel includes two end pieces that interface with the first and second layers or films. The end pieces are made from a rigid polymer material. The end pieces may include one or more ports or access passageways providing fluid pathways into and out of the container or vessel.

FIG. 15B illustrates a side view of the container or vessel of FIG. 15A.

FIG. 15C illustrates a cross-sectional view of the container or vessel taken along the line A-A of FIG. 15B.

FIG. 16A illustrates a perspective view of one embodiment of a container or vessel that incorporates a heat exchange flow path along a peripheral surface. This embodiment has multiple inlets and outlets for the heat exchanger fluid. In addition, a cap is shown attached to the flanged end with several (three) ports located therein.

FIG. 16B is a side view of the container or vessel of FIG. 16A.

FIG. 16C illustrates a cross-sectional view of the container or vessel taken along the line A-A of FIG. 16B.

FIG. 17A illustrates a perspective view of one embodiment of a container or vessel that incorporates a heat exchange flow path along a peripheral surface. This embodiment has multiple inlets and outlets for the heat exchanger fluid. In addition, a cap is shown attached to the flanged end with several (three) ports located therein. Additional ports are provided in the outer jacket.

FIG. 17B is a side view of the container or vessel of FIG. 17A.

FIG. 17C illustrates a cross-sectional view of the container or vessel taken along the line A-A of FIG. 17B.

### DETAILED DESCRIPTION OF THE ILLUSTRATED EMBODIMENTS

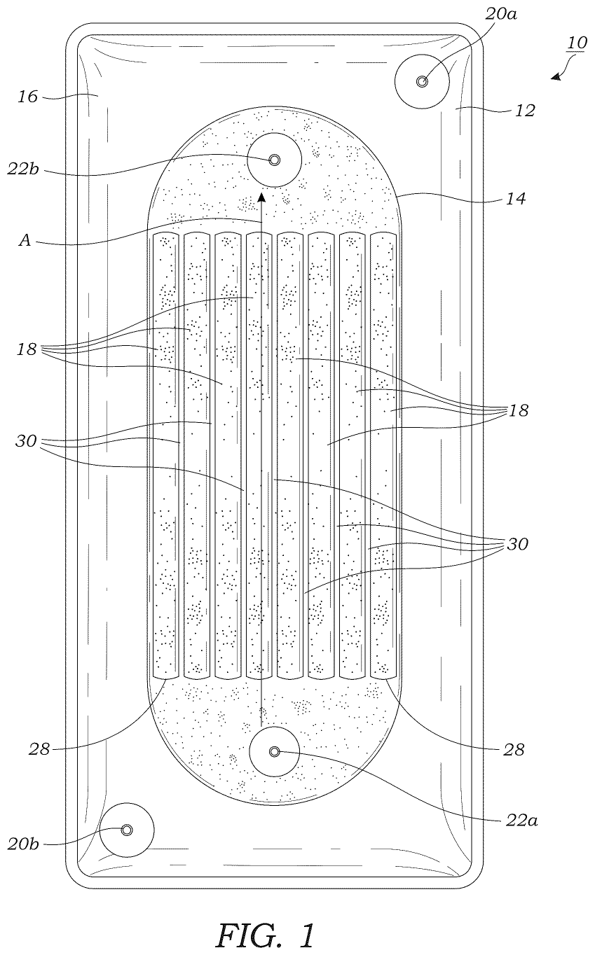

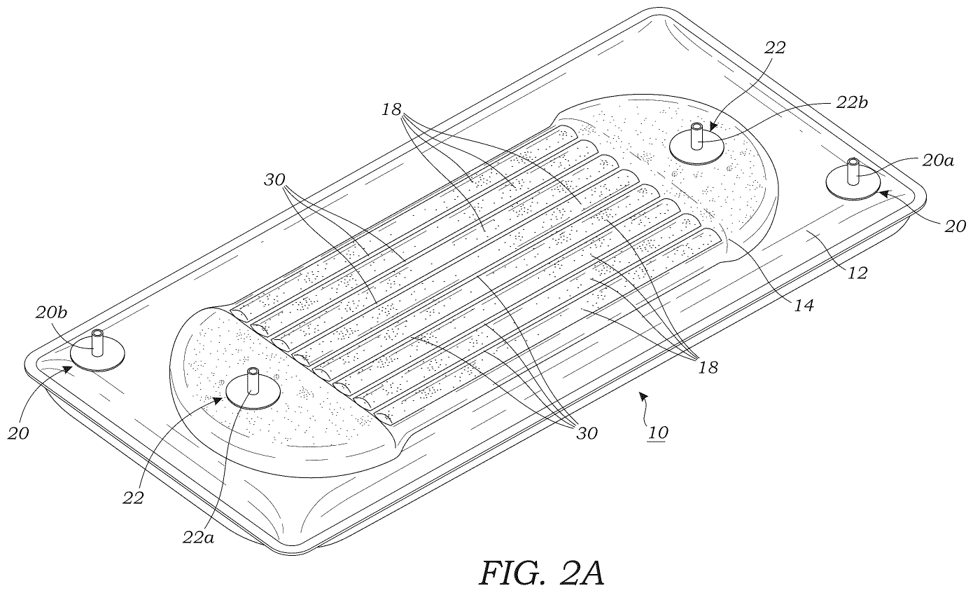

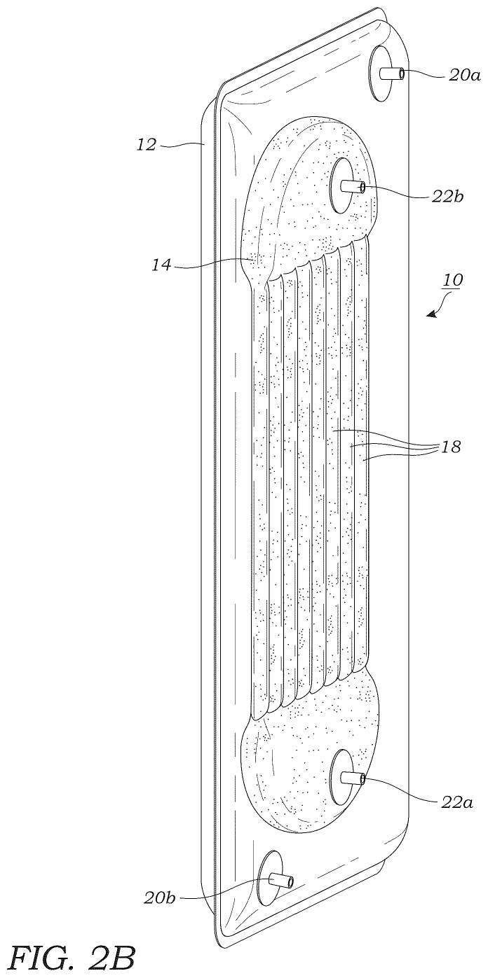

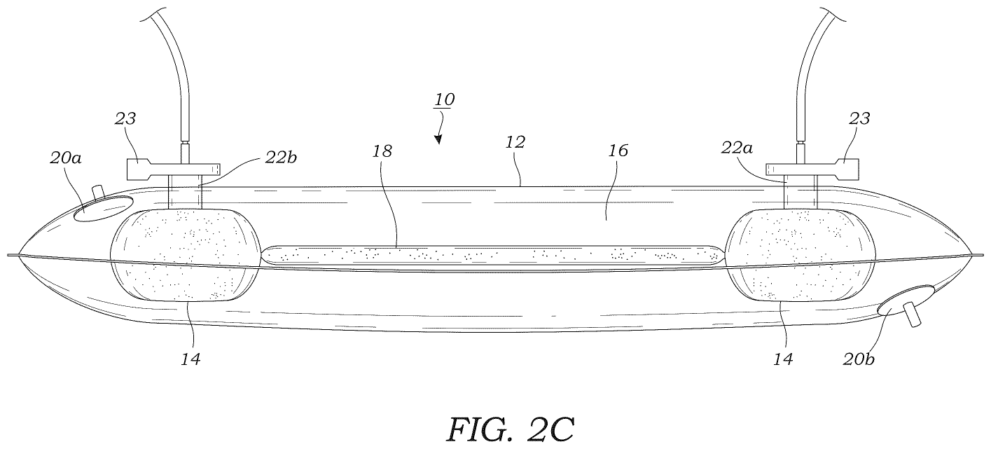

FIG. 1 illustrates one embodiment of the single use heat exchanger device 10. FIGS. 2A-2C illustrate different views of the single use heat exchanger device 10. The single use heat exchanger device 10, in this embodiment, includes an outer bag 12 and an inner bag 14. The outer bag 12 includes an interior compartment 16 that contains the inner bag 14. The outer bag 12 includes a plurality of ports 20 welded into the outer bag 12. In the illustrated embodiment, a first port 20a and a second port 20b are secured into the outer bag 12. The first port 20a and a second port 20b are secured into the outer bag 12 using, for example, a laser weld. One of the first port 20a or the second port 20b is used to flow fluid (e.g., heat exchange fluid) into the interior compartment 16 of the outer bag 12 while the other of the first port 20a or the second port 20b is used to remove fluid from the interior compartment 16 to outside of the outer bag 12.

The inner bag 14 defines a flow path 18 for carrying fluid that is isolated from the interior compartment 16. The inner bag 14 includes a plurality of ports 22 secured to the inner bag 14. In the illustrated embodiment, a third port 22a and a fourth port 22b are laser welded into the inner bag 14. One of the third port 22a or the fourth port 22b is used to flow fluid into the inner bag 14 while the other of the third port 22a or the fourth port 22b is used to remove fluid from the interior or flow path 18 of the inner bag 14. The ports 20, 22 may be polyethylene-based in some embodiments. While four (4) such ports 20a, 20b, 22a, 22b are described in this specific embodiment, it should be appreciated that additional ports 20 may be included in the outer bag 12 and/or inner bag 14.

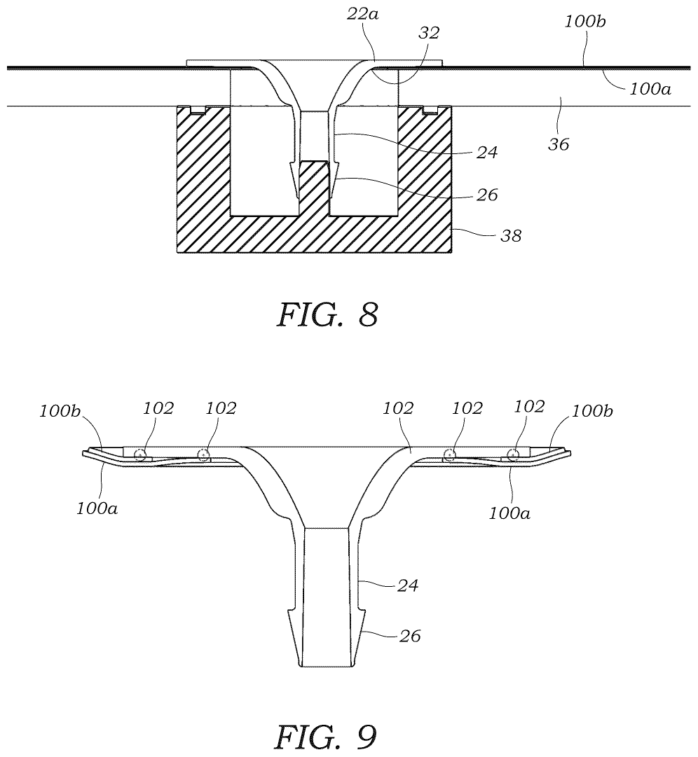

In some embodiments, the ports 20, 22 are flanged ports that have a flanged base that is secured to the inner bag 14 and/or outer bag 12 with a nipple 24 that extends outwardly from the inner bag 14 and/or outer bag 12 (seen in FIGS. 8-9). The nipple 24 may have a barbed end 26 that is used to aid in securing tubing to the respective ports 20, 22 (e.g., 20a, 20b, 22a, 22b). The ports 20, 22 may also be constructed as a variety of ends or connectors used in biopharmaceutical processes. These include hygienic connectors (e.g., FIG. 2C), barb locks, hose barbs, flanges, TC connectors, disposable aseptic connectors (DAC), and the like. Clamps 23 such as those illustrated in FIG. 2C may be used to secure feed lines, tubing, or conduit to the ports 20a, 20b, 22a, 22b. Typically, fluid is pumped continuously or semi-continuously through the outer bag 12 and inner bag 14 to control temperature of the fluid passing through the inner bag 14 (or outer bag 12 in an alternative embodiment).

As explained herein, separate fluids are contained in the outer bag 12 and the inner bag 14 and heat is exchanged between these separate fluids across the surface of the inner bag 14 or portions thereof. For example, the inner bag 14 may carry a fluid with reagents or products while the outer bag 12 carries heat exchange fluid which is used to control the temperature of the fluid with the reagents or products that is located in the inner bag 14. Control the temperature of the fluid within the inner bag 14 may include heating the reagents or products, cooling the reagents or products, or maintaining the temperature of the reagents or products at a temperature or temperature range. In some embodiments, the inner bag 14 may contain a fluid that contains cells or other organisms. The cells or other organisms may include live eukaryotic cells, prokaryotic cells, or other organisms.

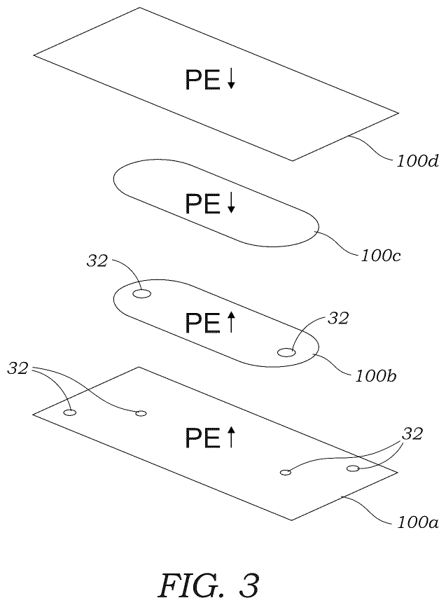

The outer bag 12 is formed from a flexible polymer film that defines the interior compartment 16. The flexible polymer film used for the outer bag 12 that defines the interior compartment 16 is made, as explained herein, from two separate layers 100a, 100d (as best seen, for example, in FIG. 3) of flexible polymer film that are secured or joined together. Various ways of joining the layers 100a, 100d are contemplated. This includes, for example, welding the layers 100a, 100d together. Various types of welding methods may be employed including heat sealing, contact-based welding and non-contacting welding operations (examples include laser welding, ultrasonic welding, heat sealing welding, and the like). In one particular embodiment, the layers 100a, 100d are joined by laser welding. Similarly, the inner bag 14 may be made, as explained herein, from two separate layers 100b, 100c (best seen in FIG. 3) of flexible polymer film that are joined together using any of the welding methods discussed herein. Each layer 100a, 100b, 100c, 100d of flexible polymer film may itself include a multi-layer film layer that includes a durable, ethylene-vinyl alcohol copolymer (EVOH) that is oriented on the outside (non-product contact) of the flexible polymer film while the inside or product contact side of the flexible polymer film is made from polyethylene (PE). Thus, in one embodiment, each layer 100a, 100b, 100c, 100d of flexible polymer film is itself a multi-layer film layer that includes EVOH that is oriented on the outside (non-product contact) of the flexible polymer film while the inside or product contact side of the flexible polymer film is made from PE. The same flexible polymer film used for the outer bag 12 can be used for the inner bag 14. Various film thicknesses are contemplated in this embodiment as well as other embodiments described herein. For example, by way of illustration and not limitation, the films may have a thickness within the range of 0.003″ to 0.020″ but thicknesses outside this range can also be used.

The inner bag 14 and the outer bag 12 are defined or formed by welds 102 that are formed at desired locations using laser welding. Specifically, in the embodiment where laser welding is used to form welds 102, a welding solution is interposed between separate layers 100a, 100b, 100c, 100d of flexible polymer film with the PE sides of the flexible polymer film facing each other and laser radiation is used to heat and weld the flexible polymer films together at predefined locations via welds 102. Similarly, the first port 20a and the second port 20b, which are polyethylene-based in one embodiment, can be welded to the flexible polymer film that makes the outer bag 12 via welds 102. The third port 22a and fourth port 22b, which are polyethylene-based in one embodiment, are also secured to the inner bag 14 and/or outer bag 12 by welds 102. As an alternative to joining the ports 20a, 20b, 22a, 22b via welds 102, an adhesive may be used to join the ports 20a, 20b, 22a, 22b to the outer bag 12 and/or inner bag 14. As still another alternative to joining the ports 20a, 20b, 22a, 22b to the outer bag 12 and/or inner bag, the ports 20a, 20b, 22a, 22b may include threads that engage with a nut or cap that engages with the threads in a mechanical connection. An optional gasket may also be employed to ensure a fluid-tight connection although in other embodiments, a fluid-tight seal may be formed without the presence of a separate gasket.

As explained herein, the inner bag 14 is formed from two separate layers of flexible polymer film that are joined (e.g., welded) together. During the welding process, an interior flow path 18 or multiple such interior flow paths 18 (e.g., FIG. 1) is/are defined that extends between the third port 22a and fourth port 22b that are located in the inner bag 14. The third port 22a and the fourth port 22b extend through the outer bag 12 and provide fluid access to and from the interior flow path 18 of the inner bag 14. The interior flow path 18 may include a plurality of separate flow paths 18 in some embodiments. For example, these separate flow paths 18 may be formed as fluid channels 28 contained in a web 30 of the inner bag 14 (five such fluid channels 28 are seen in FIG. 1). The fluid channels 28 may have a substantially circular cross-sectional shape when filled with fluid. These fluid channels 28 (seen in FIGS. 1 and 2A. 2B) are laterally separated from one another via the web 30. The web 30 may, in some embodiments, have openings formed therein to promote the circulation of fluid flow around the interior flow path 18 to improve heat transfer efficiency. Arrow A in FIG. 1 illustrates the direction of fluid flow in the interior flow path 18. In this embodiment, fluid enters the inner bag 14 via the third port 22a and passed through fluid channels 28 and exits the inner bag 14 via the fourth port 22b. The inner bag 14 is not directly connected to the outer bag 12 but is indirectly suspended or held within the outer bag 12 by the connections to the ports 20 (e.g., ports 20a, 20b).

The outer bag 12 may, in some embodiments, be designed with holes, edges, or other feature around the periphery to enable the single use heat exchanger device 10 to be hung or mounted on a wall, surface, or cart. Of course, the single use heat exchanger device 10 may also be supported or held on a horizontal surface or contained in holder that holds the same. The single use heat exchanger device 10 may operate in a generally horizontal orientation, vertical orientation, or other orientation.

To use the single use heat exchanger device 10, a fluid that contains reagents or product is pumped or otherwise flowed through the inner bag 14. Fluid enters the inner bag 14 via one of the ports 22 (e.g., one of the third port 22a or the fourth port 22b). The fluid exits the inner bag 14 via another port 22 (e.g., the other of the third port 22a or the fourth port 22b). The fluid entering the inner bag 14 may be pumped using a pump or it may be gravity fed (or other means). To exchange heat with the fluid contained in the inner bag 14, a heat exchange fluid is pumped or otherwise flowed through the outer bag 12. The heat exchange fluid enters the outer bag 12 via a port 20 (e.g., one of the first port 20a or the second port 20b). The heat exchange fluid exits the outer bag 12 via another port 20 (e.g., the other of the first port 20a or the second port 20b). Importantly, the heat exchange fluid does not come into contact with the reagent or product fluid in the inner bag 14 as the inner bag 14 serves as a barrier between the two respective fluids. The heat exchange fluid flows over the outer surface of the inner bag 14 and in particular the interior flow path(s) 18 and heat is exchanged between the two fluids. The interior flow path(s) 18 may include fluid channels 28 as described herein, provide a high surface area to improve the efficiency of heat transfer. The single use heat exchanger device 10 may be operated in a concurrent configuration in which both fluids (e.g., reagent/product and heat exchange fluid) enter on the same end or side of the single use heat exchanger device 10. Alternatively, the single use heat exchanger device 10 may be operated in a countercurrent configuration in which both fluids (e.g., reagent/product and heat exchange fluid) enter on opposite ends or sides of the single use heat exchanger device 10. In another alternative configuration, the heat exchange fluid is run through the inner bag 14 while the reagent or product is run through the outer bag 12.

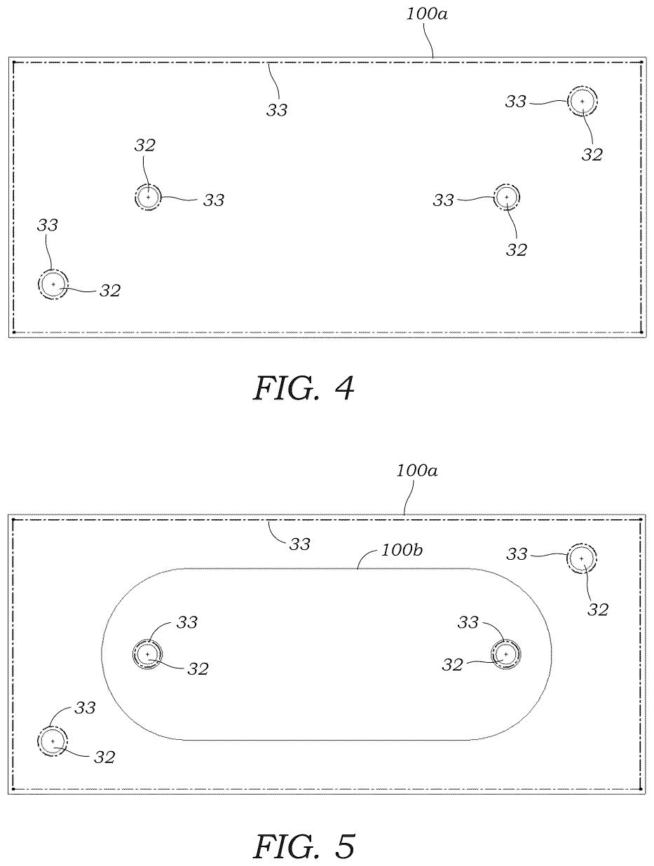

The single use heat exchanger device 10 is manufactured, in one embodiment, using a laser welding process to create the bag-in-bag construction with the inner bag 14 being located inside the outer bag 12. As explained herein, other welding processes may also be used to manufacture the single use heat exchanger device 10. With reference to FIGS. 3-9, a description will now be given of how the single use heat exchanger device 10 is manufactured. The single use heat exchanger device 10 is made from four (4) layers of flexible polymer film as described herein. The outer bag 12 is made from layer 100a and layer 100d in FIG. 3 and the inner bag 14 is made from layer 100b and layer 100c in FIG. 3. The first port 20a, second port 20b, third port 22a, and forth port 22b are provided and secured or joined to the outer bag 12 and/or inner bag 14 using an adhesive, weld, or mechanical connection as described herein. In one particular embodiment, the first port 20a, second port 20b, third port 22a, and forth port 22b are laser welded in place as described herein. Because the flexible polymer film used in layers 100a, 100b, 100c, 100d and the ports 20a, 20b, 22a, 22b are transparent to the laser light emitted by the laser welder, a welding solution is applied to the locations where the welds 102 are to be made. This is illustrated as dashed lines in FIGS. 4-6. This means that the welds 102 can be performed “through” the other layers of film where no solution is applied. Welding solution may be applied manually or automatically (e.g., with a robotic tool or the like). The welding solution absorbs radiation from the laser and causes localized heating which creates the welds 102. The laser may emit light at any number of wavelengths including those used for welding of plastic or polymer materials (e.g., infrared lasers). Of course, in non-laser welding operations there is no need for any welding solution.

Initially, the four (4) layers of flexible polymer film are cut to the desired size and apertures 32 are formed in layer 100a and layer 100b which will receive the first port 20a, second port 20b, third port 22a, and forth port 22b. It should be appreciated that the apertures 32 (and first port 20a, second port 20b, third port 22a, and forth port 22b) can be located in layer 100c and layer 100d in alternative configurations. For example, the first port 20a, second port 20b, third port 22a, and forth port 22b don't all have to be on the same side of the single use heat exchanger device 10 as some could be located on opposing sides. Next, the four (4) layers are oriented to place the PE-based sides as shown in FIG. 3 (arrows indicate PE side with up arrow indicating PE side is upper surface and down arrow indicating PE side is lower surface). This is because the laser weld is formed on the PE facing sides of the flexible polymer film.

First, layer 100a is placed on the working surface of the laser welder (e.g., surface of welding table 36 as illustrated in FIG. 8) and welding solution is placed on the PE facing surface where the profile of the weld 102 is to be made (see FIGS. 4-6). The welding table 36 may have a number of holes or apertures that pull vacuum to assist in keeping the layers stationary during the laser welding process. To this end, a layer of film that is transparent to the laser light may also be used to envelop or trap the various layers and components in a stationary state while laser welding takes place (which can be removed after welding).

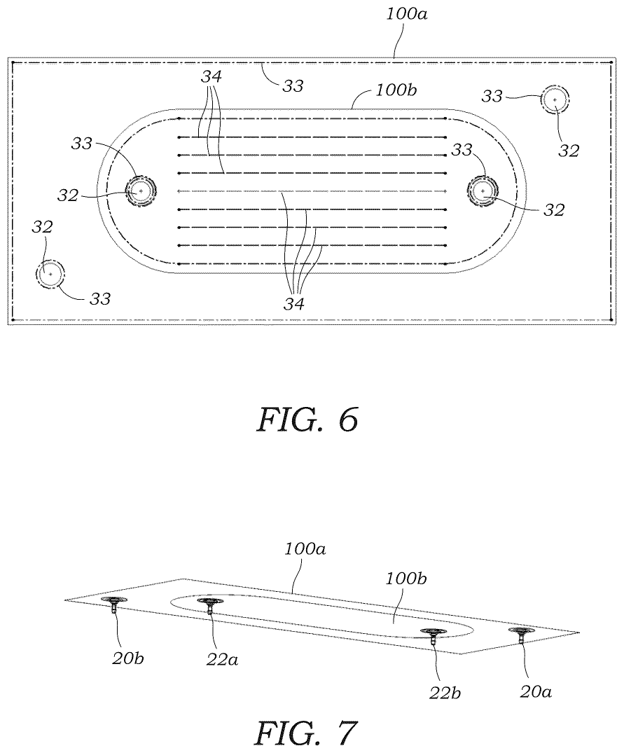

As seen in FIG. 4, the welding solution is placed around the periphery of layer 100a and around the apertures 32 (placement location of welding solution illustrated by dashed lines 33). Next, layer 100b is placed on top of layer 100a as seen in FIG. 5. The welding solution is then applied to the exposed face of layer 100b. With reference to FIG. 6, the welding solution is placed around the periphery (dashed lines 33) of layer 100b, around the apertures 32 for the third port 22a and fourth port 22b, and along separate segments 34 between the apertures 32 for the third port 22a and fourth port 22b to define separate interior flow paths 18 (FIG. 1) in the inner bag 14 (after welding operation is performed). Dashed lines 33, 34 indicate the where the welding solution is placed. The first port 20a, second port 20b, third port 22a, and fourth port 22b are then placed from above into respective apertures 32 as seen in FIG. 7. With reference to FIG. 8, the welding table 36 has recessed fixtures 38 mounted on the bottom of the welding table 36 that are secured to the bottom allowing the flanged ports 20a, 20b, 22a, 22b to sit generally flush on top of layer 100a and layer 100b.

Layer 100c and layer 100d are then placed in order on top of layer 100a and layer 100b. With all layers 100a, 100b, 100c, 100d in place, the bag weld operation is then performed using a laser welder to form the outer bag 12 (with layer 100a and layer 100d) and the inner bag 14 (layer 100b and layer 100c). The laser welder may include a robotically controlled laser head that is able to scan over the welding table 36 and irradiate the working surface in the appropriate locations where the welding solution (dashed lines in FIGS. 4-6) is located to create the welds 102. The laser welder includes may include laser head mounted on robotic gantry that is able to move in the x, y, and/or z planes.

Next, the first port 20a and the second port 20b are welded onto layer 100a using, in one embodiment, the laser welder. The first port 20a and the second port 20b may be joined or secured onto layer 100a using other types of welds 102, an adhesive, or a mechanical connection. The 22a and the fourth port 22b are then joined onto both layer 100a and layer 100b using a weld, adhesive, or a mechanical connection. To maintain the necessarily seals, the third port 22a and the fourth port 22b are welded to both the inner bag 14 (layer 100b) and the outer bag 12 (layer 100a) with two offset circular welds 102 formed around the flanges of the third port 22a and the fourth port 22b as seen in FIG. 9 (only the third port 22a is shown in FIG. 9.). After all of the ports 20a, 20b, 22a, and 22b have been welded, the single use heat exchanger device 10 can be removed from the welding table 36.

The single use heat exchanger device 10 may be subject to a sterilization operation and packaged for sale. For example, the single use heat exchanger device 10 may be subject to, in one embodiment, gamma irradiation (or another irradiation process) to sterilize the single use heat exchanger 10.

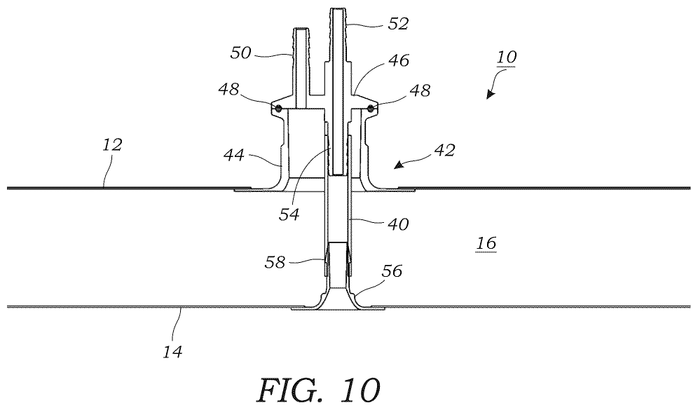

In another embodiment, and with reference to FIG. 10, the single use heat exchanger device 10 has an inner bag 14 that is suspended from the outside bag 12 via a standoff 40. In this embodiment, the outer bag 12 includes two ports 42 (one is illustrated in FIG. 10) that are used to flow fluid into the inner bag 14 and outer bag 12 and remove fluid form the inner bag 14 and outer bag 12. Each port 42 is formed from a two-piece construction that includes flanged bottom connector 44 that is welded to the outer bag 12 and provides access to the interior compartment 16. A top 46 interfaces with the flanged bottom connector 44 and a fluid-tight seal is formed between the two with an o-ring or gasket 48. A clamp or the like can be used to secure the top 46 against the flanged bottom connector 44. The top 46 includes first nipple 50 that provides fluid access to the interior compartment 16. The top 46 includes a second nipple 52 that extends from both sides of the top 46 and provides fluid access to the inner bag 14 via the standoff 40. The second nipple 52 operates as a separate fluid passageway located in the first and second ports 42.

Specifically, the portion of the second nipple 52 that extends into the flanged bottom connector 44 includes a barbed end 54 that interfaces with a segment of tubing that serves as the standoff 40. The segment of tubing that forms the standoff 40 may be flexible or rigid and have different lengths which can adjust the standoff distance between the outer bag 12 and the inner bag 14. The other end of the tubing that forms the standoff 40 is coupled to a port 56 that is secured to the inner bag 14 (two such ports 56 are used in the inner bag 14: one for fluid entry and one for fluid exit). This port 56 on the inner bag 14 may be welded thereto as illustrated in FIG. 10 and includes a barbed nipple 58 on which the tubing forming the standoff 40 is secured. In this manner, the inner bag 14 is suspended away from the inner surface of the outer bag 12 which can provide space for fluid to flow around the inner bag 14 to improve heat transfer. The two ports 42 in the outer bag 12 thus provide separate flow passageways for the fluids entering/exiting the outer bag 12 and inner bag 14.

To manufacture the embodiment of FIG. 10, the inner bag 14 with the ports 56 are formed first using laser welding as described herein. Next, the outer bag 12 is formed around the now formed inner bag 14. This can be done by interposing the formed inner bag 14 between flexible polymer film layers 100a, 100d that are used to form the outer bag 12 as previously explained. These two layers 100a, 100d of flexible polymer film may already have the ports 42 formed therein using laser welding (or the ports 42 can be secured in same process of forming the outer bag 12). Laser welding can then create the formed outer bag 12 around the inner bag 14. This is done by sealing the two layers 100a, 100d of flexible polymer film about the periphery as previously explained. Next, the tube which is used as the standoff 40 is connected between the ports 42, 56 and the respective tops 46 of the ports 42 are secured to the flanged bottom connector 44 using, for example, clamps 23 (e.g., like FIG. 2C).

It should be appreciated that various sizes and shapes of the single use heat exchanger device 10 may be manufactured using the techniques disclosed herein. In addition, the inner bag 14 may have one or more interior flow paths 18 and may have any number of configurations. These include straight channels 28, curved channels 28, angled channels 28, and other configurations. Likewise, the number of interior flow paths 18 may vary in different designs. This includes a single interior flow path 18 or multiple flow paths 18. Generally, the number, configuration, and dimensions of the interior flow path(s) 18 is designed to improve the overall surface area that is exposed to the heat exchange fluid within the outer bag 12.

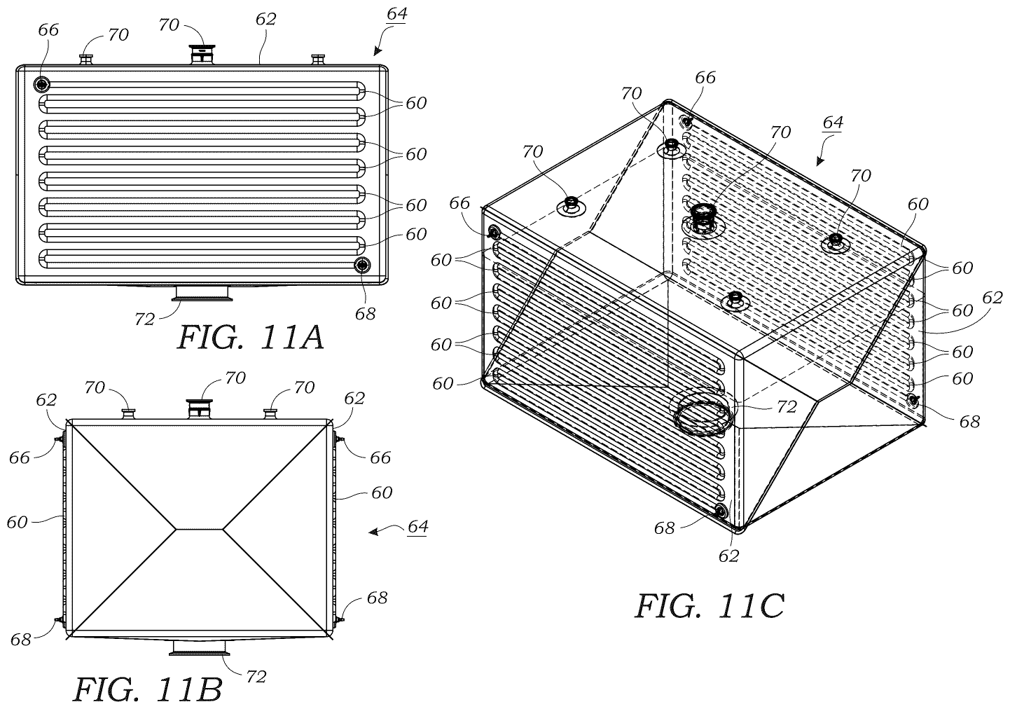

FIGS. 11A-11C illustrate an alternative embodiment of a flexible bag or container 64 that incorporates a heat exchange flow path 60 into one or more sides or surfaces 62. The flexible bag or container 64 is thus a single use product that incorporates heat exchange functionality therein. The heat exchange flow path 60 is formed into one or more surfaces 62 of the flexible bag or container 64. The heat exchange flow path 60 has an s-coil shape that traverses the sides of the flexible bag or container 64. The heat exchange flow path 60 includes a series of s-shaped coils or curves created by welding select locations of the outer surface of the flexible bag or container 64 to a flexible polymer film layer (e.g., layer 100a, 100b, 100c, 100d) that defines the heat exchange flow path 60. In this embodiment, two such sides or surfaces 62 of a flexible bag or container 64 (best seen in FIG. 11C) have separate heat exchange flow paths 60 integrated therein. Of course, more or fewer surfaces 62 may have a heat exchange flow path 60. Each heat exchange flow path 60 includes an inlet port 66 and an outlet port 68 through which flows heat exchange fluid. The inlet port 66 and outlet port 68 may include any type of port described herein or typically used in pharmaceutical/biopharmaceutical applications. The heat exchange fluid thus flows along the s-coil shaped heat exchange flow path(s) 60 from the inlet port(s) 66 to the outlet port(s) 68. In this embodiment, the flexible bag or container 64 contains one or more inlet ports 70 and one or more outlet ports 72 (for passage of product, reagents, buffers, waste, and the like) for fluid going into and out of the interior volume of the flexible bag or container 64. In this particular embodiment, the flexible bag or container 64 has five (5) inlet ports 70 on the top of the flexible bag or container 64 and a single outlet port 72 located at the bottom of the flexible bag or container 64. It should be appreciated that any number of inlet ports 70 and outlet ports 72 may be used. Moreover, the size(s) and location of the inlet ports 70 and outlet ports 72 may vary. Further, the location of these ports 70, 72 may be located in different surfaces. The one or more inlet ports 70 and one or more outlet ports 72 may include any type of port described herein or typically used in pharmaceutical and/or biopharmaceutical applications. This includes, for example, flanged port connectors, nipple connectors, and other aseptic connectors.

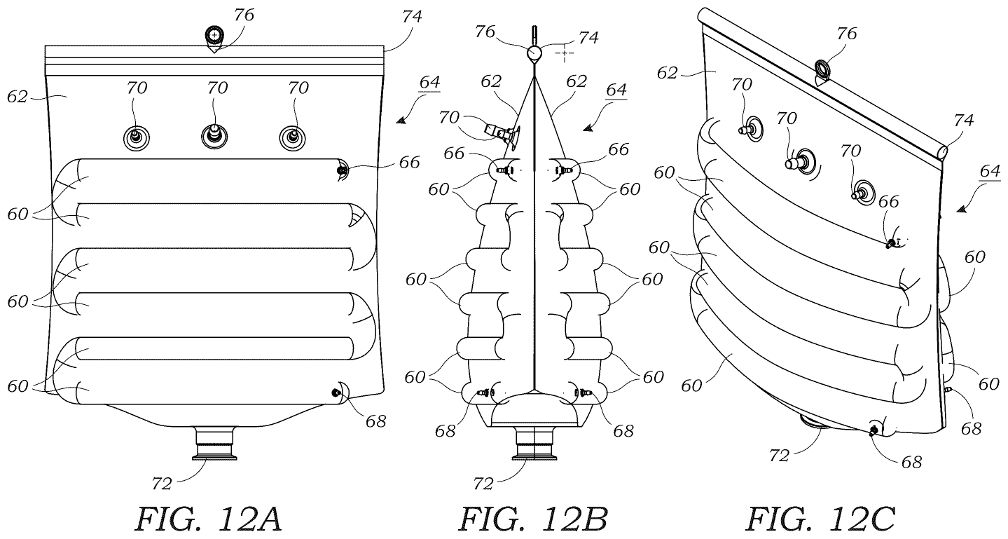

FIGS. 12A-12C illustrate an alternative embodiment of a flexible bag or container 64 that incorporates a heat exchange flow path 60 into one or more sides or surfaces 62. The flexible bag or container 64 is thus a single use product that incorporates heat exchange functionality therein. The heat exchange flow path 60 is formed into one or more surfaces 62 of the flexible bag or container 64. The flexible bag or container 64 in this embodiment is a tapered flexible bag or container 64 that has a common edge with surfaces extending therefrom that flare outward to define the interior volume of the flexible bag or container 64. The heat exchange flow path 60 has an s-coil shape that traverses the sides 62 of the flexible bag or container 64. The heat exchange flow path 60 includes a series of s-shaped coils or curves created by welding select locations of the outer surface of the flexible bag or container 64 to a flexible polymer film layer (e.g., layers 100b or 100c) that defines the heat exchange flow path 60. In this embodiment, two opposing sides or surfaces 62 of a flexible bag or container 64 have separate heat exchange flow paths 60 integrated therein. Each heat exchange flow path 60 includes an inlet port 66 and an outlet port 68 through which flows heat exchange fluid. The inlet port 66 and outlet port 68 may include any type of port described herein or typically used in pharmaceutical/biopharmaceutical applications. In addition, the locations of the inlet port 66 and the outlet port 68 may vary. For example, the locations of the inlet port 66 and outlet port 68 may be reversed. The heat exchange fluid thus flows along the s-coil shaped heat exchange flow path 60 from the inlet port 66 to the outlet port 68. In this embodiment, the flexible bag or container 64 contains one or more additional inlet ports 70 and one or more outlet ports 72 the allow for fluid to enter/exit the interior of the flexible bag or container 64 (e.g., for passage of product, reagents, buffers, waste, and the like). In this particular embodiment, the flexible bag or container 64 has three (3) inlet ports 70 on the top of the flexible bag or container 64 and a single outlet port 72 located at the bottom of the flexible bag or container 64. It should be appreciated that any number of inlet ports 70 and outlet ports 72 may be used. Moreover, the size(s) and location of the inlet ports 70 and outlet ports 72 may vary. Further, the location of these ports 70, 72 may be located in different surfaces. The one or more inlet ports 70 and one or more outlet ports 72 may include any type of port described herein or typically used in pharmaceutical and/or biopharmaceutical applications. This includes, for example, flanged port connectors, nipple connectors, and other aseptic connectors. The flexible bag or container 64 of FIGS. 12A-12C may include a tubular opening 74 that is configured to receive a hanger 76 (e.g., rod) that is used to hang the flexible bag or container 64 in a vertical orientation.

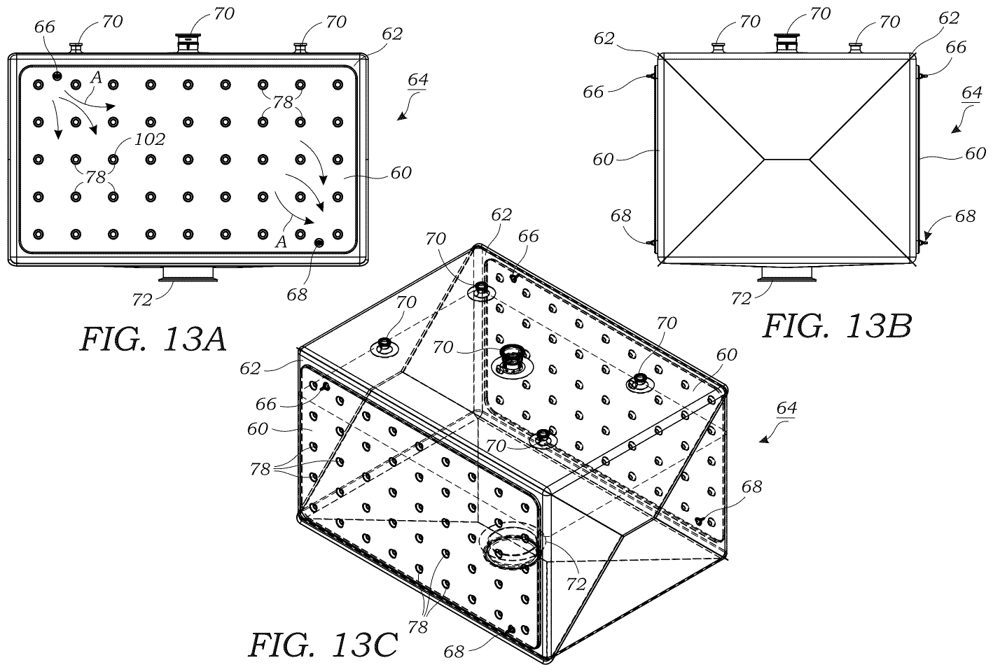

FIGS. 13A-13C illustrate an alternative embodiment of a flexible bag or container 64 that incorporates a heat exchange flow path 60 into one or more sides or surfaces 62. The flexible bag or container 64 is thus a single use product that incorporates heat exchange functionality therein. The heat exchange flow path 60 is formed into one or more surfaces 62 of the flexible bag or container 64. The heat exchange flow path 60, in this embodiment, includes a dimpled surface formed by dimples 78 created by welding select locations of the outer surface of the flexible bag or container 64 to a flexible polymer film (e.g., a layer like layer 100a) that defines the heat exchange flow path 60 that extend across the sides of the flexible bag or container 64. The dimples 78 may include circular welds 102 populated between the flexile bag or container 64 and the flexible film used to define the heat exchange flow path 60. In this embodiment, two opposing sides or surfaces of a flexible bag or container 64 have separate heat exchange flow paths 60 integrated therein. Each heat exchange flow path 60 includes an inlet port 66 and an outlet port 68 through which flows heat exchange fluid. The inlet port 66 and outlet port 68 may include any type of port described herein or typically used in pharmaceutical and/or biopharmaceutical applications. In addition, the locations of the inlet port 66 and the outlet port 68 may vary. For example, the locations of the inlet port 66 and outlet port 68 may be reversed. The heat exchange fluid thus flows between the dimples 78 and along the heat exchange flow path 60 from the inlet port 66 to the outlet port 68. This fluid flow is illustrated by arrows A in FIG. 13A. The dimples 78 aid in distributing or spreading out the heat exchange fluid along the heat exchange flow path 60. In this embodiment, the flexible bag or container 64 contains one or more additional inlet ports 70 and one or more outlet ports 72 for the passage of fluid into and out of the flexible bag or container 64 (e.g., for passage of product, reagents, buffers, waste, and the like). In this particular embodiment, the flexible bag or container 64 has five (5) inlet ports 70 on the top of the flexible bag or container 64 and a single outlet port 72 located at the bottom of the flexible bag or container 64. It should be appreciated that any number of inlet ports 70 and outlet ports 72 may be used. Moreover, the size(s) and location of the inlet ports 70 and outlet ports 72 may vary. Further, the location of these ports 70, 72 may be located in different surfaces 62. The one or more inlet ports 70 and one or more outlet ports 72 may include any type of port described herein or typically used in pharmaceutical and/or biopharmaceutical applications. This includes, for example, flanged port connectors, nipple connectors, and other aseptic connectors.

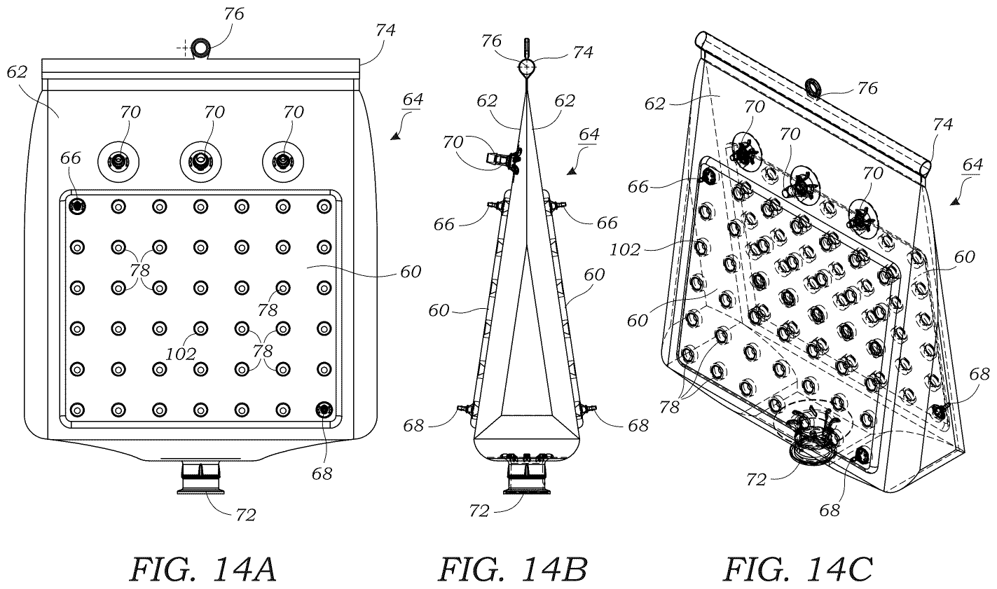

FIGS. 14A-14C illustrate an alternative embodiment of a flexible bag or container 64 that incorporates a heat exchange flow path 60 into one or more sides or surfaces 62. The flexible bag or container 64 is thus a single use product that incorporates heat exchange functionality therein. The heat exchange flow path 60 is formed into one or more surfaces 62 of the flexible bag or container 64. The flexible bag or container 64, in this embodiment, is a tapered flexible bag that has a common edge with surfaces extending therefrom that flare outward to define the interior volume of the flexible bag or container 64. The heat exchange flow path 60 includes a dimpled surface formed by dimples 78 created by welding select locations of the outer surface of the flexible bag or container 64 to a flexible polymer film (e.g., like layer 100a) that defines the heat exchange flow path 60) that extend across the sides of the flexible bag or container 64. The dimples 78 may include circular welds 102 populated between the flexile bag or container 64 and the flexible film layer (e.g., layer 100a) used to define the heat exchange flow path 60. In this embodiment, two opposing sides or surfaces 62 of a flexible bag or container 64 have separate heat exchange flow paths 60 integrated therein. Each heat exchange flow path 60 includes an inlet port 66 and an outlet port 68 through which flows heat exchange fluid. The inlet port 66 and outlet port 68 may include any type of port described herein or typically used in pharmaceutical and/or biopharmaceutical applications. In addition, the locations of the inlet port 66 and the outlet port 68 may vary. For example, the locations of the inlet port 66 and outlet port 68 may be reversed. The heat exchange fluid thus flows along the dimpled heat exchange flow path 60 from the inlet port 66 to the outlet port 68. In this embodiment, the flexible bag or container 64 contains one or more additional inlet ports 70 and one or more outlet ports 72 for the passage of fluid into and out of the flexible bag or container 64 (e.g., for passage of product, reagents, buffers, waste, and the like). In this particular embodiment, the flexible bag 64 has three (3) inlet ports 70 on the top of the flexible bag or container 64 and a single outlet port 72 located at the bottom of the flexible bag or container 64. It should be appreciated that any number of inlet ports 70 and outlet ports 72 may be used. Moreover, the size(s) and location of the inlet ports 70 and outlet ports 72 may vary. Further, the location of these ports 70, 72 may be located in different surfaces 62. The one or more inlet ports 70 and one or more outlet ports 72 may include any type of port described herein or typically used in pharmaceutical or biopharmaceutical applications. This includes, for example, flanged port connectors, nipple connectors, and other aseptic connectors. The flexible bag or container 64 of FIGS. 14A-14C may include a tubular opening 74 that is configured to receive a hanger 76 (e.g., rod) that is used to hang the flexible bag or container 64 in a vertical orientation.

It should be appreciated that other configurations of the heat exchanger flow path 60 beyond the s-coil and dimpled configurations illustrated in FIGS. 11A-11C, 12A-12C, 13A-13D, and 14A-14D may be formed on one or more surfaces 62 of the flexible bag or container 64. The heat exchanger flow path(s) 60 may each further have any number of flow paths (e.g., a single flow path, two or more flow paths) in any number of configurations and geometries using structures like channels 28 as described herein. In addition, the heat exchanger flow path(s) 60 may be formed on the outside surface 62 of the flexible bag or container 64 or on an inside surface 62 of the flexible bag or container 64. In some embodiments, the heat exchanger flow path 60 may extend over more than one surface (e.g., multiple sides).

To make the embodiments of FIGS. 11A-11C, 12A-12C, 13A-13D, and 14A-14D, the heat exchanger flow path 60 is formed by using a flexible polymer film layer such as layers 100a, 100b, 100c, 100d described herein that is laser welded to define the heat exchanger flow path 60 on one or more surfaces of the flexible bag or container 64. This may be on the outside surface 62 of the flexible bag or container 64 but may also be located on the inside surface 62 of the flexible bag or container 64. In one embodiment, the heat exchanger flow path 60 is formed on a flexible bag or container 64 that has already been manufactured. Alternatively, the heat exchanger flow path 60 may be formed as part of the manufacturing process used to create the flexible bag or container 64. Preferably, the flexible polymer film that is laser welded onto the surface(s) of the flexible bag or container 64 is made from the same material as the flexible bag or container 64 or has a facing contact surface that is made from the same material as the flexible bag or container 64. This enables more robust welds that resist delamination. During manufacturing, the inlet port 66 and outlet port 68 may be interposed between the flexible polymer film and the surface 62 of the flexible bag or container 64 and the respective ports 66, 68 passing through apertures formed in the flexible polymer film. Weld solution is applied around the respective ports 66, 68. around the exterior or perimeter of the flexible polymer film and at selected locations to define the heat exchanger flow path(s) 60 and is welded to the flexible bag or container 64.

To use the heat exchange functionality of the flexible bag or containers 64 of FIGS. 11A-11C, 12A-12C, 13A-13D, and 14A-14D, heat exchange fluid is pumped or otherwise flowed into the inlet port(s) 66. This may be assisted by one or more pumps connected to the inlet port(s) 66 via conduits or lines. Fluid then leaves the heat exchanger flow path(s) 60 via the outlet port(s) 68. Fluid containing reagents or products are loaded into or flowed into the interior compartment of the flexible bag or container 64 and temperature is controlled (e.g., heated, cooled, maintained) by the heat exchange fluid. Fluid containing reagents or products may be pumped or flowed continuously through the flexible bag or container 64 via ports 70, 72 or fluid may be maintained in the flexible bag or container 64 for a period of time (e.g., operating in batch mode). Various conduits or fluid lines may be connected to the various ports 20a, 20b, 22a, 22b, 42, 56, 66, 68, 70, 72 described herein. The ports 20a, 20b, 22a, 22b, 42, 56, 66, 68, 70, 72 may also be coupled to other devices such as pumps (e.g., pump secured to outlet port 72). For example, the pump or combination pump/mixer device disclosed in International Patent Publication No. WO 2021/158448 (PCT/US2021/015917), which is incorporated herein by reference, may be used. The flexible bag or container 64 is preferably a single use device. It should be appreciated the flexible bag or container 64 may come in any number of shapes and sizes and one or more surfaces 62 have incorporated therein the heat exchanger flow path 60.

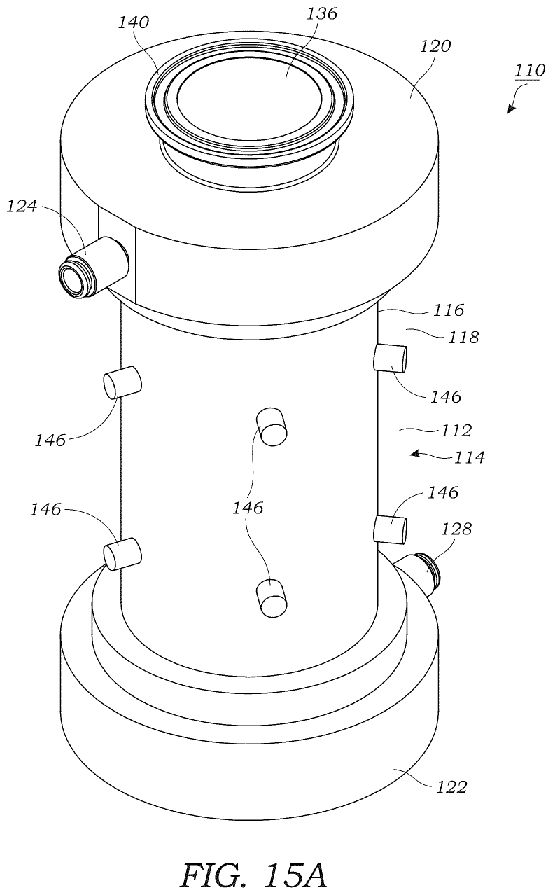

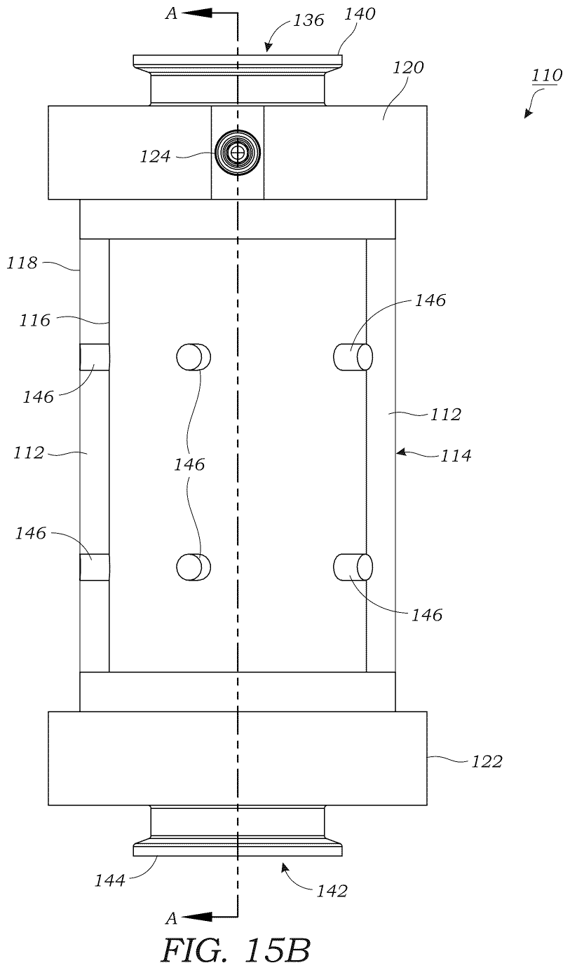

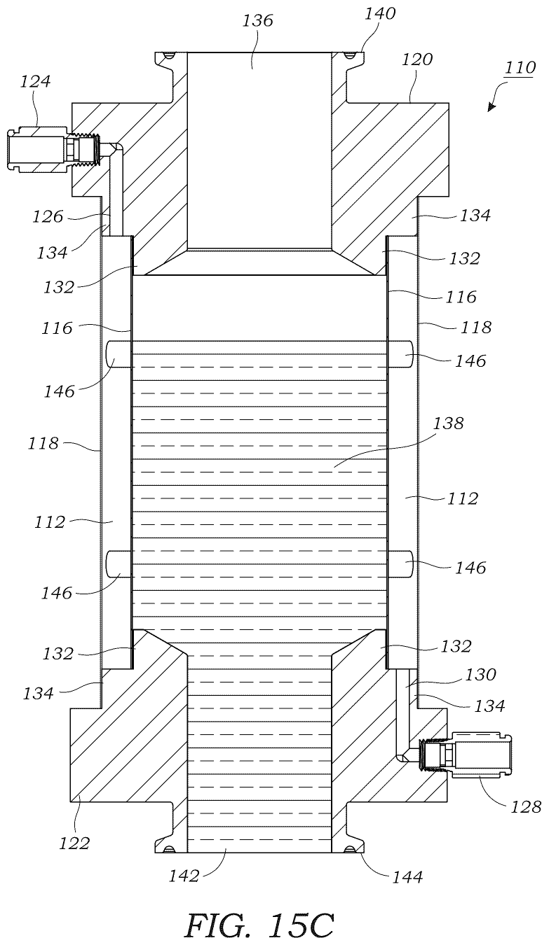

FIGS. 15A-15C illustrates views of one embodiment of a container or vessel 110 that incorporates a heat exchange flow path 112 along a peripheral surface 114. In this embodiment, the peripheral surface 114 of the container or vessel 110 is a cylindrically-shaped surface that incorporates an inner jacket 116 that defines an inner, product-contacting surface of the container or vessel 110. An outer jacket 118 is disposed radially outward of the inner jacket 116 and the heat exchange flow path 112 is located in the space between the inner jacket 116 and the outer jacket 118. The container or vessel 110 includes a first fitting 120 located at one end of the container or vessel 10 that interfaces with ends of the inner jacket 116 and the outer jacket 118. A second fitting 122 is located at an opposing end of the container or vessel 110 that also interfaces with ends of the inner jacket 116 and the outer jacket 118. The first and second fittings 120, 122 function as end pieces of the container or vessel 110. A heat exchange fluid inlet port 124 is located on the first fitting 120 and communicates with fluid passageway 126 (FIG. 15C) disposed in the first fitting 120 that communicates with the annular space between the inner jacket 116 and the outer jacket 118 that defines the heat exchange flow path 112. A heat exchange fluid outlet port 128 is located on the second fitting 122 and communicates with fluid passageway 130 disposed in the second fitting 122 that communicates with the annular space between the inner jacket 116 and the outer jacket 118 that defines the heat exchange flow path 112.

With reference to FIG. 15C, the first fitting 120 includes a first radial surface 132 where the inner jacket 116 is secured to the first fitting 120. The inner jacket 116 may be bonded or welded to the first radial surface 132. For example, as one example of welding, the inner jacket 116 may be welded to the first radial surface 132 using rotary welding. The first fitting 120 includes a second radial surface 134 where the outer jacket 118 is secured to the first fitting 120. The second radial surface 134 is located radially outward with respect to the first radial surface 132. The outer jacket 118 may be bonded or welded to the second radial surface 134 as described above.

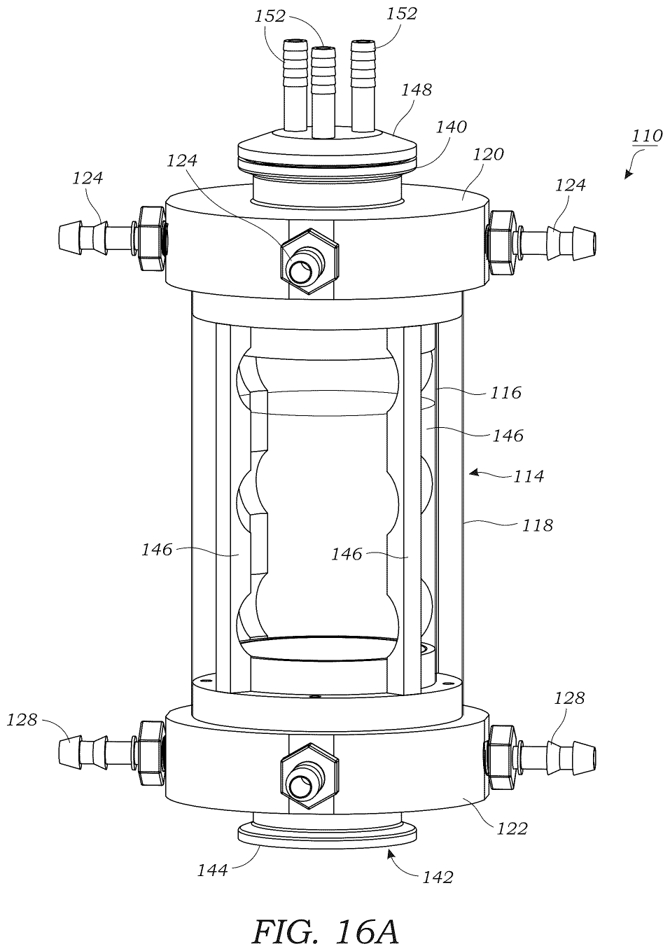

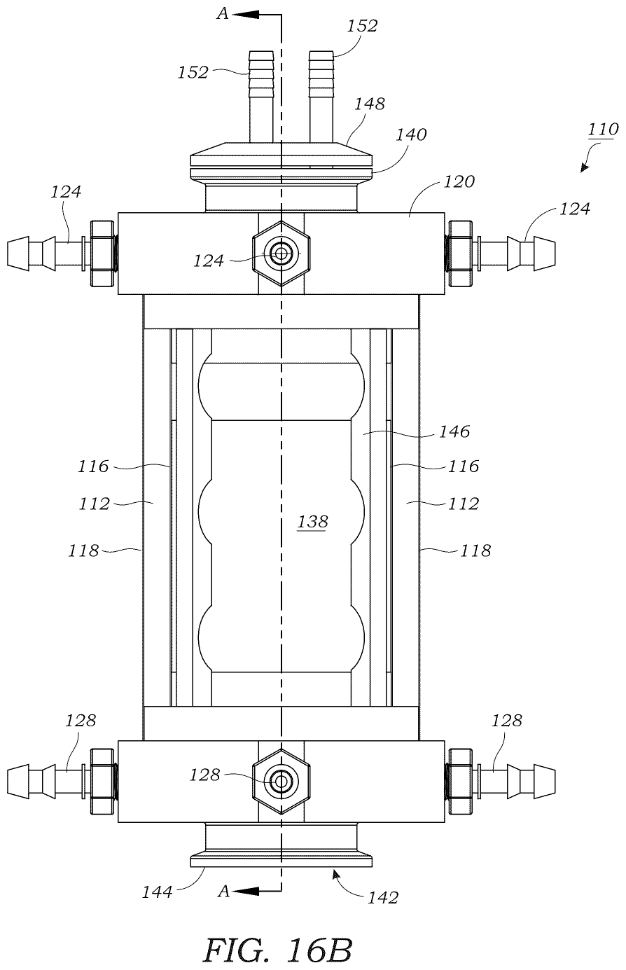

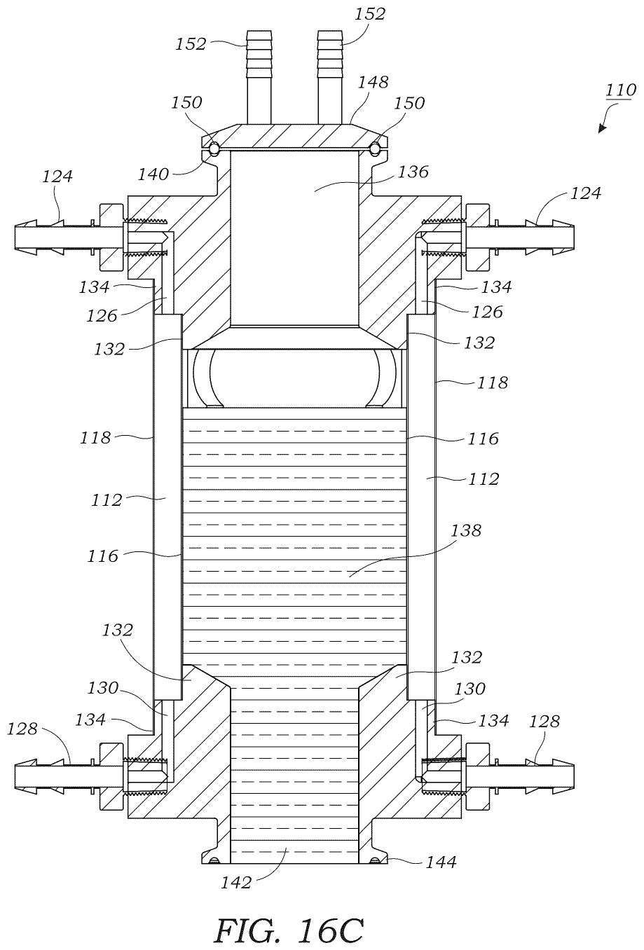

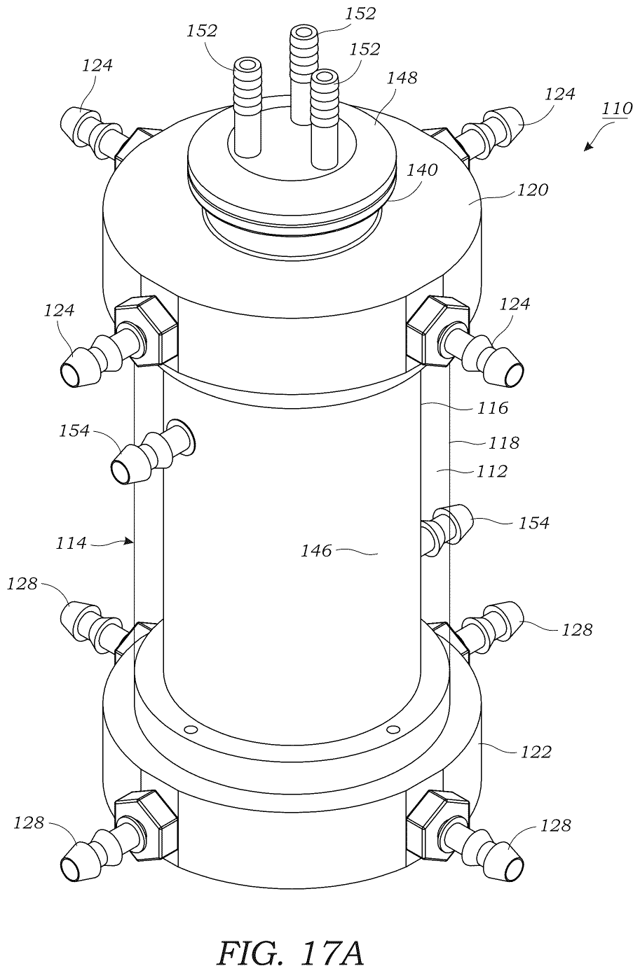

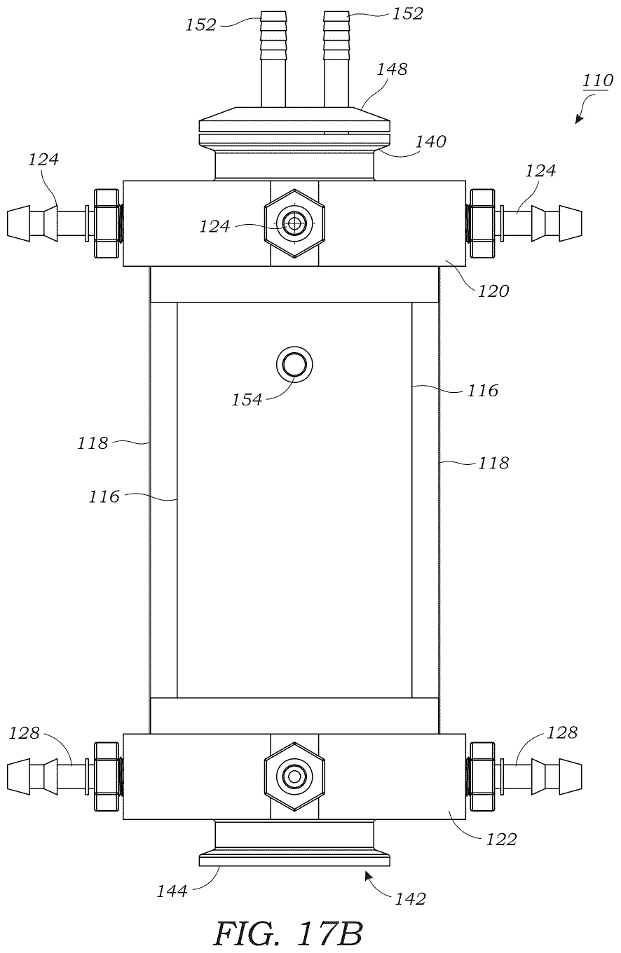

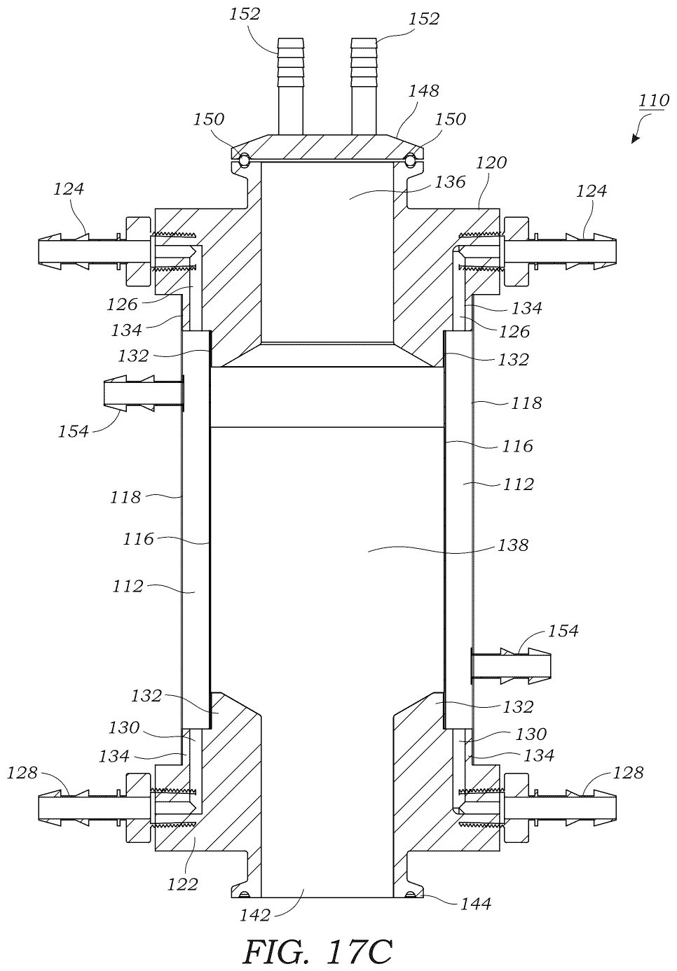

In the embodiment of FIGS. 15A-15C, the first fitting 120 includes an inlet 136 that communicates with the interior space 138 of the container or vessel 110 as seen in FIG. 15C. The inlet 136 may include a flanged end 140 that may be used, for example, as part of a hygienic claim such as Tri-Clamp sanitary fitting connection. The flanged end 140 enables the container or vessel 110 to be connected to another component, process, or the like. For example, one or more feed lines may be coupled to the flanged end 140 via a mating cap 148 (seen in FIGS. 16A-16C, 17A-17C). The second fitting 122 includes an outlet 142 (FIGS. 15B, 15C) that communicates with the interior space 138 of the container or vessel 110. The outlet 142 may include a flanged end 144 that may be used, for example, as part of a hygienic claim such as a Tri-Clamp sanitary fitting connection as described above. The flanged end 144 enables the opposing end of the container or vessel 110 to be connected to another component, process, or the like. For example, the outlet 142 may be coupled to a pump device that is used to pump fluid out of and/or into the interior space 138 of the container or vessel 110.

During operation, heat exchange fluid enters the heat exchange fluid inlet port 124 and enters the heat exchange flow path 112. Product fluid enters the interior space 138 via the inlet 136. Heat exchange occurs across the inner jacket 116 between the heat exchange fluid and the product fluid contained in the interior space 138 of the container or vessel 110. As with other embodiments described herein, this may include heating the product fluid, cooling the product fluid, or maintaining the temperature of the product fluid at a certain temperature setpoint or temperature range. The product fluid may be pumped into or removed from the container or vessel 110 via a pump. The product fluid may flow through the container or vessel 110 continuously or semi-continuously. The container or vessel 110 could also operate in batch mode where product fluid is contained therein for a period of time and then removed from the interior space 138 using, for example, a pump.

The heat exchange fluid inlet port 124 and the heat exchange fluid outlet port 128 may be integrally formed with the first fitting 120 and second fitting 122, respectively. Alternatively, the heat exchange fluid inlet port 124 and the heat exchange fluid outlet port 128 may be connected to the first fitting 120 and second fitting 122, respectively using, for example, known means of connection. This includes, for example, threaded ends on the heat exchange fluid inlet port 124 and the heat exchange fluid outlet port 128 that mate with corresponding threaded receiving regions formed in the first fitting 120 and the second fitting 122. Other known connections such as push-to-connect and the like may also be used. The heat exchange fluid inlet port 124 and the heat exchange fluid outlet port 128 may include a barbed end or the like so that tubing or conduit may be readily connected to the container or vessel 110. It should be appreciated that any number of port types may be used for the heat exchange fluid inlet port 124 and the heat exchange fluid outlet port 128.

With reference to FIGS. 15A-15C, the container or vessel 110 includes optional spacers 146 that are located in the heat exchange flow path 112. The spacers 146 are connected at one end to the inner jacket 116 and at the opposing end to the outer jacket 118. Multiple such spacers 146 may be located around the periphery of the container or vessel 110 as illustrated. The spacers 146 aid in establishing the heat exchange flow path 112 and may also aid in disrupting or mixing the heat exchange fluid that is passing therethrough.

The first fitting 120 and the second fitting 122 are preferably made from a rigid polymer material suitable for biological, pharmaceutical, or other industrial applications. Examples includes, polyethylene, polypropylene, and ethylene tetrafluoroethylene (ETFE) although other suitable polymer materials may be used. The inner jacket 116 and the outer jacket 118 are also made from a polymer material suitable for biological, pharmaceutical, or other industrial applications including polyethylene, polypropylene, and ethylene tetrafluoroethylene (ETFE) although other suitable polymer materials may be used. The inner jacket 116 and outer jacket 118 may be made of the same or different material used for the first fitting 120 and the second fitting 122. The material used for the inner jacket 116 and outer jacket 118 should be weld-compatible with the material used for the first fitting 120 and the second fitting 122.

In some embodiments, the inner jacket 116 and/or the outer jacket 118 may be made from a single layer or film. In other embodiments, the inner jacket 116 and/or the outer jacket 118 may include multiple layers or films that form a composite or laminate structure. For multiple layers or films may aid to combat or prevent fluid migration. Even though the inner jacket 116 and/or outer jacket 118 are formed by layer(s) or film(s), the overall container or vessel 110 is a substantially rigid structure.

FIGS. 16A-16C illustrate an alternative embodiment of a container or vessel 110. Similar components to those of the embodiment of FIGS. 15A-15C use similar reference numbers. In this embodiment, there are a plurality of heat exchange fluid inlet ports 124 and a plurality of heat exchange fluid outlet ports 128. For example, in the illustrated embodiment, there are four (4) heat exchange fluid inlet ports 124 and four (4) heat exchange fluid outlet ports 128. Each port 124, 128 includes corresponding fluid passageways 126, 128 located in the first and second fittings 120, 122, respectively. Of course, fewer or more ports 124, 128 may be used in other embodiments. In addition, in this embodiment, a cap 148 is illustrated secured to the flanged end 140. A fluid-tight seal is provided between the cap 148 and the flanged end 140 by the gasket 150. A hygienic clamp (not shown) can be used to hold the cap 148 and the flanged end 140 securely together. In this embodiment, the cap 148 has a plurality of ports 152 that extend through the body of the cap 148 and provide fluid access into the interior space 138 of the container or vessel 110. FIGS. 16A-16C illustrate three (3) such ports 152, however, more or fewer ports 152 may be used. The ports 152 may have barbed or ridged ends as shown. It should be appreciated that other types of ports 152 may be used to provide fluid access to the container or vessel 100. For example, this may include hygienic ports, press-on ports, Luer ports/fittings (e.g., Luer-lock or Luer-slip), or the like. The embodiment of FIGS. 16A-16C also includes one or more spacers 146 between the inner jacket 116 and the outer jacket 118. The spacers 146 may include ribs that extend longitudinally along the length of the inner jacket 116 and outer jacket 118 and include cutouts that enable the passage of fluid.

FIGS. 17A-17C illustrate an alternative embodiment of a container or vessel 110. Similar components to those of the embodiment of FIGS. 16A-16C use similar reference numbers. This embodiment is similar to the embodiment of FIGS. 16A-16C except that the spacers 146 are omitted. In addition, one or more ports 154 are located on the outer jacket 118. The one or more ports 154 may be bonded or welded into the outer jacket 118. The port(s) 154 may be used as inlets and/or outlets. For example, in one embodiment, one of the ports 154 is used as an inlet for heat exchanger fluid while the other port 154 is used as an outlet for heat exchanger fluid. It should also be appreciated that, in other embodiments, the heat exchange fluid inlet port(s) 124 and/or the heat exchange fluid outlet port(s) 128 may be omitted entirely. In this alternative configuration, the outer jacket 118 includes at least one port 154 that functions as an inlet for the heat exchange fluid and at least one port 154 that functions as an outlet for the heat exchange fluid.

It should be appreciated that the containers or vessel 110 may incorporate of s-shaped coils or curves in the heat exchange flow path 112 as disclosed herein. In addition, the containers or vessel 110 may incorporate dimples between the inner jacket 116 and outer jacket 118 as disclosed herein. Optional spacers 146 may be included between the inner jacket 116 and outer jacket 118.

The flexible bag, containers, or vessels 64, 110 described herein may hold a variety of volumes in the interior space 138. For example, larger containers or vessels 110 may contain a several liters of product fluid. In other embodiments, smaller bags, containers, or vessels 64, 110 with interior volume/space 138 volumes of 1 liter or less may be used. Examples of volumes include 750 mL or less, 500 mL or less, 250 mL or less, 150 mL or less, 100 mL or less, 50 mL or less, 25 mL or less. The bags, containers, or vessels 64, 110 may be utilized in any number of applications and processes. For example, the bags. containers, or vessels 64, 110 may be used as bioreactors. The bags, containers, or vessels 64, 110 may also be used in personalized medicine applications. For example, the bags, containers, or vessels 64, 110 may be used to grow therapeutic cells that are utilized in gene therapy applications. These cells may include patient derived (i.e., autologous) cells or allogenic cells.

While embodiments of the present invention have been shown and described, various modifications may be made without departing from the scope of the present invention. Aspects of any particular embodiment described herein may also be used with other embodiments. The invention, therefore, should not be limited, except to the following claims, and their equivalents.

1. A single use heat exchanger device comprising:
an outer bag formed from a flexible polymer film and defining an interior compartment, the outer bag having a first port and a second port disposed therein to provide fluid access to and from the interior compartment; and
an inner bag formed from a flexible polymer film and defining an interior flow path that extends between a third port and a fourth port that extend through the outer bag and provide fluid access to and from the interior flow path of the inner bag.

2. The single use heat exchanger device of claim 1, wherein the interior flow path comprises a plurality of separate flow paths or channels disposed along a portion of the inner bag between the third port and the fourth port.

3. The single use heat exchanger device of claim 2, wherein the plurality of separate flow paths or channels have a substantially circular cross-sectional shape.

4. The single use heat exchanger device of claim 2, wherein the plurality of separate flow paths or channels are connected to one other via a web.

5. The single use heat exchanger device of claim 4, wherein the web comprises a plurality of openings formed therein.

6. The single use heat exchanger device of claim 2, wherein the plurality of separate flow or channels paths are substantially straight.

7. The single use heat exchanger device of claim 2, wherein the plurality of separate flow paths or channels are curved.

8. The single use heat exchanger device of claim 2, wherein the plurality of separate flow paths or channels are spaced apart from one another via a web or spacer.

9. The single use heat exchanger device of claim 1, wherein the third port and the fourth port are each welded to the inner bag and the outer bag at separate weld points.

10. The single use heat exchanger device of claim 1, wherein the outer bag is formed from a layer of flexible polymer film that is welded to another layer of flexible polymer film.

11. The single use heat exchanger device of claim 1, wherein the inner bag is formed from a layer of flexible polymer film that is welded to another layer of flexible polymer film.

12. The single use heat exchanger device of claim 1, wherein the inner bag is suspended from an inner surface of the outer bag via the third port and the fourth port.

13. The single use heat exchanger device of claim 1, wherein the first port, second port, third port, and fourth port comprise flanged ports having respective flanges welded to an inner surface of the outer bag and/or inner bag.

14. A method of using the single user heat exchanger device of claim 1, comprising:
flowing a reagent or product fluid stream into the third port or fourth port; and
flowing a heat exchange fluid into the first port or second port.

15. The method of claim 14, wherein a flow direction of the heat exchange fluid is opposite to the flow direction of the reagent or product fluid stream.

16. A single use heat exchanger device comprising:
an outer bag formed from a flexible polymer film and defining an interior compartment, the outer bag having a first port and a second port disposed therein to provide fluid access to and from the interior compartment; and
an inner bag formed from a flexible polymer film and defining an interior flow path that extends between a third port and a fourth port that provide fluid access to and from the interior flow path of the inner bag, wherein the third port and the fourth port are suspended by a standoff from a separate fluid passageway located in the first port and second port.

17. The single use heat exchanger device of claim 16, wherein the standoff is a tube.

18. The single use heat exchanger device of claim 16, wherein the first port and the second port comprise a two-part port having a bottom portion and a top portion, wherein an o-ring or gasket is interposed between the bottom portion and the top portion.

19. The single use heat exchanger device of claim 18, wherein the top portion comprise a first nipple and second nipple defining separate flow paths and wherein the second nipple defines the separate fluid passageway.

20. A single use heat exchanger device comprising:
a flexible bag or container having one or more surfaces and defining an interior volume, the flexible bag or container having one or more inlet ports and one or more outlet ports disposed therein to provide fluid access to and from the interior volume wherein one or more of the surfaces of the flexible bag or container comprises a heat exchanger flow path contained thereon, wherein each heat exchanger flow path comprises a heat exchange fluid inlet port and outlet port.

21. The single use heat exchanger device of claim 20, wherein the heat exchanger flow path comprises an s-coil shape.

22. The single use heat exchanger device of claim 20, wherein the heat exchanger flow path comprises dimpled surface.

23. The single use heat exchanger device of claim 20, wherein the heat exchanger flow path comprises a flexible polymer film disposed on an inner surface of the flexible bag or container.

24. The single use heat exchanger device of claim 20, wherein the heat exchanger flow path comprises a flexible polymer film disposed on an outer surface of the flexible bag or container.

25. The single use heat exchanger device of claim 20, wherein the flexible bag or container has a plurality of surfaces and a plurality of heat exchanger flow paths are disposed on the plurality of surfaces.

26. A container or vessel having an integrated heat exchanger comprising:
an inner jacket comprising one or more polymer films or layers;
an outer jacket comprising one or more polymer films or layers;
a first fitting located at one end of the inner jacket and the outer jacket and comprising an inlet, the first fitting further comprising a first radial surface secured to the inner jacket and a second radial surface secured to the outer jacket;
a second fitting located at an opposing end of the inner jacket and the outer jacket and comprising an outlet, the second fitting further comprising a first radial surface secured to the inner jacket and a second radial surface secured to the outer jacket;
at least one heat exchange fluid inlet port disposed in the first fitting and communicating with a fluid passageway contained in the first fitting that fluidically communicates with a space between the inner jacket and the outer jacket; and
at least one heat exchange fluid outlet port disposed in the second fitting and communicating with a fluid passageway contained in the second fitting that fluidically communicates with the space between the inner jacket and the outer jacket.

27. The container or vessel of claim 26, wherein the inner jacket and the outer jacket are cylindrical shaped.

28. The container or vessel of claim 26, further comprising spacers disposed between the inner jacket and the outer jacket.

29. The container or vessel of claim 26, comprising a plurality of heat exchange fluid inlet ports disposed in the first fitting and a plurality of heat exchange fluid outlet ports disposed in the second fitting.

30. The container or vessel of claim 26, wherein the inlet of the first fitting and/or the outlet of the second fitting comprise a flanged end.

31. The container or vessel of claim 30, further comprising a cap secured to the flanged end of the first fitting and having one or more ports extending therethrough.

32. The container or vessel of claim 26, further comprising one or more inlet ports on the outer jacket and in fluid communication with the space between the inner jacket and the outer jacket.

33-38. (canceled)
